(19) United States
(12) Patent Application Publication
(10) Pub. No.: US 2021/0085742 A1
(43) Pub. Date: Mar. 25, 2021

(54) FRUIT EXTRACT AND USES THEREOF

(71) Applicant: THE UNIVERSITY OF NOTTINGHAM, Nottinghamshire (GB)

(72) Inventors: Andrew BOTTLEY, Leicestershire (GB); Graham SEYMOUR, Leicestershire (GB); Natalie CHAPMAN, Leicestershire (GB)

(21) Appl. No.: 16/634,459

(22) PCT Filed: Jul. 27, 2018
(86) PCT No.: PCT/GB2018/052113

§ 371 Date: Jan. 27, 2020

(30) Foreign Application Priority Data
Jul. 28, 2017 (GB) 1712155.9
Mar. 8, 2018 (GB) 1803734.1

(51) Int. Cl. A61K 36/81; A61K 31/7032; A61K 31/198; A61K 31/047; A61K 31/194; A61K 31/355; A61K 31/7004; A61P 25/28; A61P 35/00; A61K 9/00; A61K 9/20

(52) U.S. Cl. CPC A61K 36/81; A61K 31/7032; A61K 31/198; A61K 31/047; A61K 31/194; A61K 9/0056; A61K 31/7004; A61P 25/28; A61P 35/00; A61K 9/0095; A61K 9/205; A61K 31/355

(57) ABSTRACT

An extract of a fruit from the solanaceae family containing bioavailable MGDG, wherein MGDG has the formula of Formula I. (I), and nutraceutical or pharmaceutical products comprising the extract and uses thereof.

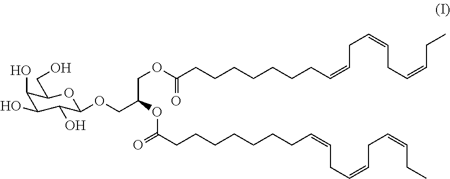 [SMILES: CC/C=C\C/C=C\C/C=C\CCCCCCCC(=O)OC[C@H](COC1OC(CO)C(O)C(O)C1O)OC(=O)CCCCCCC/C=C\C/C=C\C/C=C\CC]

The present invention relates to fruit extracts containing bioavailable monogalactosyl diacylglycerol (MGDG), and to the use of these extracts in the treatment and/or prevention of a number of conditions, including one or more of autism spectrum disorder (ASD), cancer, anxiety, inflammation and/or for improving sports cognition. The invention also provides a method for the optimal harvesting of fruit, in particular fruit of the solanaceae family such as tomatoes or peppers, to maximise the levels of bioavailable MGDG recovered, and to fruit extracts manufactured by this method and to nutraceutical and pharmaceutical products containing the fruit extracts.

In recent years there has been an ever increasing interest in the use of naturally sourced products as nutraceuticals; that is, to use products derived from food sources which have extra health benefits in addition to the basic nutritional value found in food. A particular interest has been placed on the tomato fruit which has assumed the status of a ‘functional food’ due to the association between its consumption and a reduced likelihood of certain types of cancers and cardiovascular disease. However, the nutraceutical value of tomatoes can be affected by the cultivation conditions and the time of harvest. The present invention identifies MGDG as a compound of nutraceutical and pharmaceutical importance and teaches how to harvest tomatoes to ensure recovery of maximum levels of bioavailable MGDG.

Further the invention demonstrates that naturally occurring MGDG is effective in the treatment and/or prevention of a number of conditions, including one or more of autism spectrum disorder (ASD), cancer and inflammation, and/or for improving sports cognition, in particular for improving psychomotor function.

The present invention provides for an extract of a fruit from the solanaceae family, such as a tomato, containing bioavailable MGDG, wherein MGDG has the formula of Formula I.

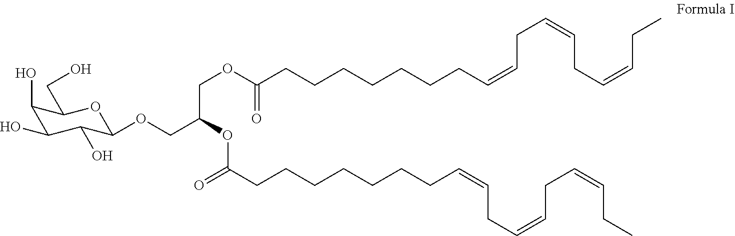 [SMILES: CC/C=C\C/C=C\C/C=C\CCCCCCCC(=O)OC[C@H](COC1OC(CO)C(O)C(O)C1O)OC(=O)CCCCCCC/C=C\C/C=C\C/C=C\CC]

The MGDG in the extract is naturally occurring MGDG.

Preferably the extract is a tomato extract.

In an embodiment of the invention, a kilogram of harvested unprocessed fruit may comprise at least 50 mg of bioavailable MGDG, preferably at least 60 mg or more, preferably at least 80 mg or more, preferably at least 100 mg or more. Preferably a kilogram of harvested fruit comprises between about 50 mg and 200 mg of bioavailable MGDG, or between 60 mg and 150 mg of bioavailable MGDG.

The extract may be provided as a pulp, a liquid, a paste or in the form of a dried powder. The dried powder may be produced by any suitable method, for example evaporation, filtration or drying, such as freeze drying. The fruit may be freeze dried with added beta-cyclodextrin to protect the MGDG in the GI tract.

A pulp may refer simply to homogenised fruit. A liquid may refer to a filtered homogenate.

The invention may also provide a pulped fruit product comprising at least 50 mg, 60 mg, 80 mg, 100 mg or more of bioavailable MGDG per kilogram of pulp. Preferably the pulped fruit is tomato fruit.

The invention may also provide a fruit paste produced from pulp according to the invention. The paste may be produced by evaporating water from the pulp. The paste may contain at least 1 mg MGDG per 1 g of paste.

The invention may also provide a fruit powder produced from pulp or paste according to the invention. The powder may be produced by freeze drying. The powder may contain at least about 2 to about 10 mg MGDG per 1 g of powder.

The extract of the invention may further comprise one or more of the following metabolites in the ratio listed:
a ratio glutamic acid to lutein of 0.00006 to 1 or less
a ratio of malic acid to lutein of 0.00069 to 1 or less
a ratio of alpha-tocopherol to lutein of 1.52 to 1 or less
a ratio of glutamic acid to citric acid of 0.02 to 1 or less
a ratio of malic acid to citric acid of 0.22 to 1 or less
a ratio of alpha-tocopherol to citric acid of 505 to 1 or less
a ratio of glutamic acid to pentose of 3.2 to 1 or less
a ratio glutamic acid to MGDG of 0.0005 to 1 or less
a ratio of malic acid to MGDG of 0.005 to 1 or less
a ratio of alpha-tocopherol to MGDG of 13 to 1 or less.

Extracts according to the invention may be used in various formulations, such as in nutraceutical and pharmaceutical products, accordingly the invention further provides nutraceutical and/or pharmaceutical products comprising a fruit extract, preferably a tomato extract, according to the invention.

The fruit extracts of the invention may be formulated for oral administration. As such, they can be formulated as solutions, drinks, suspensions, syrups, tablets, capsules, lozenges and snack bars. The extracts may be formulation as a powder for rehydration before use. Such formulations may be prepared in accordance with methods well known to the art.

For example, the extract may be formed into a syrup or other solution for administration orally, for example as a health drink. One or more excipients selected from sugars, vitamins, flavouring agents, colouring agents, preservatives and thickeners may be included in such syrups or solutions. Tonicity adjusting agents such as sodium chloride, or sugars, may be added to provide a solution of a particular osmotic strength, for example an isotonic solution. One or more pH-adjusting agents, such as buffering agents may also be used to adjust the pH to a particular value, and preferably maintain it at that value. Examples of buffering agents include sodium citrate/citric acid buffers and phosphate buffers.

Alternatively, the extract may be dried (e.g. by spray drying or freeze drying) and the dried product formulated in a solid or semi solid dosage form, for example as a tablet, lozenge, capsule, powder, granulate or gel.

Compositions containing the extracts may be prepared without any additional components. Alternatively, they may be prepared by adsorbing on to a solid support; for example a sugar such as sucrose, lactose, glucose, fructose, mannose or a sugar alcohol such as xylitol, sorbitol or mannitol; or a cellulose derivative. Other particularly useful adsorbents include starch-based adsorbents such as cereal flours for example wheat flour and corn flour.

For tablet formation, the extract may typically be mixed with a diluent such as a sugar, e.g. sucrose and lactose, and sugar alcohols such as xylitol, sorbitol and mannitol; or modified cellulose or cellulose derivative such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener may be added, for example ammonium glycyrrhizinate or an artificial sweetener such as aspartame, or sodium saccharinate.

The extracts may also be formulated as powders, granules or semisolids for incorporation into capsules. When used in the form of powders, the extracts may be formulated together with any one or more of the excipients defined above in relation to tablets, or can be presented in an undiluted form. For presentation in the form of a semisolid, the dried extracts can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cod liver oil, herring oil, etc. Such extracts may be filled into capsules of either the hard gelatine or soft gelatine type or made from hard or soft gelatine equivalents, soft gelatine or gelatine-equivalent capsules being preferred for viscous liquid or semisolid fillings.

Extracts according to the invention may also be provided in a powder form for incorporation into snack food bars for example fruit bars, nut bars, and cereal bars. For presentation in the form of snack food bars, the extracts may be admixed with any one or more ingredients selected from dried fruits such as sun-dried tomatoes, raisins and sultanas, groundnuts or cereals such as oats and wheat.

Extracts according to the invention may also be provided in a powder form for reconstitution as a solution. As such they can also contain soluble excipients such as sugars, buffering agents such as citrate and phosphate buffers, and effervescent agents formed from carbonates, e.g. bicarbonates such as sodium or ammonium bicarbonate, and a solid acid, for example citric acid or an acid citrate salt.

In one preferred embodiment, an extract according to the invention is provided in powder form optionally together with a preferred solid (e.g. powdered) excipient for incorporation into capsules, for example a hard gelatine capsule.

A solid or semisolid dosage form of the present invention may contain up to about 15 mg of bioavailable naturally sourced MGDG, for example up to about 12 mg.

The extract may be presented as a food supplement or food additive, or may be incorporated into foods, for example functional foods or nutraceuticals. A food supplement refers to a food product which provides physiological benefits or protects of prevents against disease. The food supplement may be a drink.

The extract of the invention may be presented in the form of unit dosage forms containing a defined amount of bioavailable naturally sourced MGDG. Such unit dosage forms may be selected so as to achieve a desired level of biological activity. For example, a unit dosage form can contain an amount of up to about 20 mg (dry weight) of bioavailable naturally sourced MGDG, more typically up to about 15 mg, for example between about 1 and about 5 mg, or between about 2 mg and about 20 mg. The unit dosage forms may comprise about 1, 2, 3, 4, 5, 6, 7, 8, 9, 10, 11, 12, 13, 14, 15, 16, 17, 18, 19, 20 or more mg of MGDG.

The unit dosage form may be a drink, a powder to be added to a drink or other foodstuff (such as a yoghurt or a snack bar) or a tablet/capsule for ingestion.

The extracts of the invention can be included in a container, pack or dispenser together with instructions for administration.

In a further aspect the invention provides a method of treating and/or preventing one or more of autism spectrum disorder (ASD), cancer and inflammation, and/or for improving sports cognition, in particular for improving psychomotor function, comprising administering a effective amount of a fruit extract according to any other aspect of the invention. Preferably the fruit extract is from tomatoes.

In another aspect the invention provides the use of a fruit extract according to any other aspect of the invention for use in the preparation of a medicament for treating and/or preventing one or more of autism spectrum disorder (ASD), cancer and inflammation, and/or for improving sports cognition, in particular for improving psychomotor function. Preferably the fruit extract is from tomatoes.

In another aspect, the invention provides a fruit extract according to any other aspect of the invention for use in treating and/or preventing one or more of autism spectrum disorder (ASD), cancer and inflammation, and/or for improving sports cognition, in particular for improving psychomotor function. Preferably the fruit extract is from tomatoes.

For the treatment or prevention of specific diseases and conditions, the quantity of the bioavailable MGDG to be administered to a patient per day will depend upon the particular condition or disease under treatment and its severity, and ultimately it will be at the discretion of the physician/subject. The amount administered however will typically be a non-toxic amount effective to prevent or treat the condition in question.

If the bioavailable MGDG, preferably obtained from tomatoes, is be used as a cancer prophylactic in a human a daily dose of at least 0.05 mgs of bioavailable MGDG per kg of human may be administered. Preferably a daily dose of between about 0.05 and about 0.5 mg of bioavailable MGDG per kg of human is administered. This dose may be administered in a single or multiple dose.

The inventors have shown that a dose of approximately 5.5 mgs of bioavailable MGDG would be sufficient to achieve a 30% inhibition in the growth of cancer cells in a 60kg human. This equates to a dose of about 0.092 mgs of bioavailable MGDG per kg of human. Alternatively, consumption of a slightly higher dose, such as a daily of a dose of approximately 11 mgs of bioactive is sufficient to achieve a 50% inhibition in the growth of cancer cells. This equates to a dose of about 0.183 mgs of bioavailable MGDG per kg of human.

If the bioavailable MGDG is to be used in a smaller mammal such as dog (for example an 8 kg dog), administration of a daily dose of about 3 mgs of MGDG may be sufficient to achieve a 30% inhibition in the growth of cancer cells. The MGDG may provided as a supplement added to pet food, or it may be provided in the pet food.

If the bioavailable MGDG is to be used with even smaller mammal such as a mouse (for example an 20 g mouse) a daily dose of about 60 micrograms of bioavailable MGDG may be sufficient to achieve a 50% inhibition of the growth of cancer cells.

If the bioavailable MGDG, preferably obtained from tomatoes, is be used to treat an autism spectrum disorder, to enhance visual IQ and/or to enhance sports cognition, in particular for improving psychomotor function, in a human a daily dose of at least about 6 mgs of bio-available MGDG may be sufficient, for example, to ameliorate the symptoms of autism spectrum disorder. The MGDG may be provided in the form of pulped fruit, preferably pulped tomatoes, which are added to a daily dose fruit drink.

If the bioavailable MGDG obtained from tomatoes is be used to as an anti-inflammatory a dose of about 10 μg of bioavailable MGDG is sufficient to elicit an ˜80% reduction of IL-6 inflammatory activity in IL-1α (interleukin-1alpha)+TNFα (Tumour Necrosis Factor alpha) treated cells. Therefore a daily dose of about 7.5 mgs of bioavailable MGDG would be sufficient to achieve anti-inflammatory activity in a 60 kg human. In an 8 kg dog, this would equate to a daily dose of 2 mg dose of bioavailable MGDG.

Typically MGDG is “locked” in the thylakoid membrane of chloroplasts and has very limited bioavailability. However, for a limited period during tomato ripening the MGDG is unlocked from the chloroplast membrane and at this point it is bioavailable, it is at this point the tomatoes need to be harvested to allow the maximum levels of MGDG to be recovered. The MGDG may be bioavailable for only a few hours, or the window may be a few days

According to another aspect, the present invention provides a method of obtaining bioavailable MGDG from fruit comprising harvesting the fruit when the MGDG is in the bioavailable form, wherein the method comprises the steps of:
determining the level in the fruit of two or more of the following metabolites glutamic acid, lutein, malic acid, alpha-tocopherol, pentose and citric acid;
comparing the relative levels of the two or more metabolites;
comparing the observed relative levels to known relative levels and harvesting the fruit when relative levels indicative of high levels of bioavailable MGDG are observed.

Preferably the method is used to obtain MGDG from tomatoes.

If one or more of the following ratios are observed in a fruit, preferably a tomato, the fruit may be considered ready to pick and the levels of bioavailable MGDG can be expected to be at least about 60 to about 300 mg per kg of fruit, preferably at least about 100 to about 300 mg per kg of fruit:
a ratio of glutamic acid to lutein of 0.00006 to 1 or less
a ratio of malic acid to lutein of 0.00069 to 1 or less
a ratio of alpha-tocopherol to lutein of 1.52 to 1 or less
a ratio of glutamic acid to citric acid of 0.02 to 1 or less
a ratio of malic acid to citric acid of 0.22 to 1 or less
a ratio of alpha-tocopherol to citric acid of 505 to 1 or less
a ratio of glutamic acid to pentose of 3.2 to 1 or less
a ratio glutamic acid to MGDG of 0.0005 to 1 or less
a ratio of malic acid to MGDG of 0.005 to 1 or less
a ratio of alpha-tocopherol to MGDG of 13 to 1 or less.

Alternatively, or additionally, if one or more of the following absolute levels are observed in a fruit, preferably a tomato, the fruit may be considered ready to pick and the levels of bioavailable MGDG can be expected to be at least about 60 to about 300 mg per kg of fruit, preferably at least about 100 to about 300 mg per kg of fruit:
level of glutamic acid per gram of about 0.003 μg/mg or less
level of malic acid per gram of about 0.03 μg/mg or less
level of alpha-tocopherol per gram of about 79 μg/mg or less

In order to select fruit at the optimum point for harvest they may be selected on the fragrance or odour produced by volatile organic compounds released by the fruit. The optimal odour fingerprint for a fruit containing the equivalent of at least about 60 mg of bioavailable MGDG per kg of fruit, preferably at least 100 mg/kg, may be determined by assaying for one or more of the following volatile organic compounds: beta-ionone, hexanal, beta-damascenone, 1-penten-3-one, 2-methylbutanal, trans-2-hexenal, isobutylthiazole, 1-Nitro-2-phenylethane, trans-2-heptenal, phenylacetaldehyde, 6-methyl-5-hepten-2-one, cis-3-hexanol, 2-Phenylethanol, 3-methylbutanol and methyl salicylate.

The volatile compounds may be detected by using any suitable technique. One method would be to use an electronic nose system to evaluate the aroma or odour of fruit. This system uses a sensor array to evaluate all of the chemical constituents present in an aroma, it then coverts this to an electrical signal, which is assembled to form a distinct pattern (Electronic Aroma Signature Pattern) (Baietto et al Sensors 2015, 15, 899-931).

In another embodiment, the invention provides a method of selecting a fruit for harvesting, the fruit may be a tomato or another fruit of the solanaceae family, wherein a kilogram of harvested fruit contains at least 60 mg of bioavailable MGDG, wherein the method comprises:
obtaining fruit;
determining the odour fingerprint by assaying for one or more of the following volatile organic compounds: beta-ionone, hexanal, beta-damascenone, 1-penten-3-one, 2-methylbutanal, trans-2-hexenal, isobutylthiazole, 1-Nitro-2-phenylethane, trans-2-heptenal, phenylacetaldehyde, 6-methyl-5-hepten-2-one, cis-3-hexanol, 2-Phenylethanol, 3-methylbutanol and methyl salicylate; and
selecting those fruit with the optimal odour fingerprinting.

In an alternative embodiment, the invention provides a method of harvesting tomatoes, or another fruit of the solanaceae family, wherein a kilogram of harvested fruit contains at least 60 mg of bioavailable MGDG, wherein the method comprises:
obtaining fruit;
determining one or more of the following:
   ratio glutamic acid to lutein
   ratio of malic acid to lutein
   ratio of alpha-tocopherol to lutein
   ratio of glutamic acid to citric acid
   ratio of malic acid to citric acid
   ratio of alpha-tocopherol to citric acid
   ratio of glutamic acid to pentose
   ratio glutamic acid to MGDG
   ratio of malic acid to MGDG
   ratio of alpha-tocopherol to MGDG
using the ratio to identify fruit with the maximum levels of bioavailable MGDG
selecting the fruit with maximum levels of bioavailable levels MGDG.

The ratios of the various metabolites referred to above may be determined by assaying for one or more of the following volatile organic compounds: beta-ionone, hexanal, beta-damascenone, 1-penten-3-one, 2-methylbutanal, trans-2-hexenal, isobutylthiazole, 1-Nitro-2-phenylethane, trans-2-heptenal, phenylacetaldehyde, 6-methyl-5-hepten-2-one, cis-3-hexanol, 2-Phenylethanol, 3-methylbutanol and methyl salicylate.

The selected fruit identified by a method of the invention may then be processed, for example homogenised to produce a pulp which may be further processed before use.

The method of the invention is intended to allow fruit with the equivalent of at least 60 mg of bioavailable MGDG per kg of fruit to be identified and harvested. Preferably the fruits when harvested contain the equivalent of at least about 60 mg of bioavailable MGDG per kg of fruits, more preferably the tomatoes when harvested contain the equivalent of at least 70, 80 90, 100, 100, 120, 130, 140, 150 mg or more of bioavailable MGDG per kg of fruits. Preferably the fruits when harvested contain the equivalent of about 150 mg to 300 mgs of bioavailable MGDG per kg of fruits.

Once a fruit has been determined to be ready for harvesting by the method of the invention the fruit may be harvested to any suitable method. Once harvested the fruit may be processed. In one embodiment the fruit may be homogenised to produce a fruit homogenate the homogenate may then be filtered through a filter having a molecular weight cutoff of 1000 Da to produce a filtrate; and the filtrate may then be collected to provide an extract. In some embodiments, the method may also comprise the steps of freeze-drying the homogenate to produce a freeze-dried homogenate. The freeze-dried homogenate may be dissolved in water and used or frozen for storage, or the freeze-dried homogenate may be used directly or stored frozen as a powder.

The invention also provides for an extract of fruit obtained by the method of the invention. Preferably the fruit are tomatoes.

The skilled man will appreciate that preferred features of any one embodiment and/or aspect of the invention may be applied to all other embodiments and/or aspects of the invention.

There now follows by way of example only a detailed description of the present invention with reference to the accompanying drawings, in which:

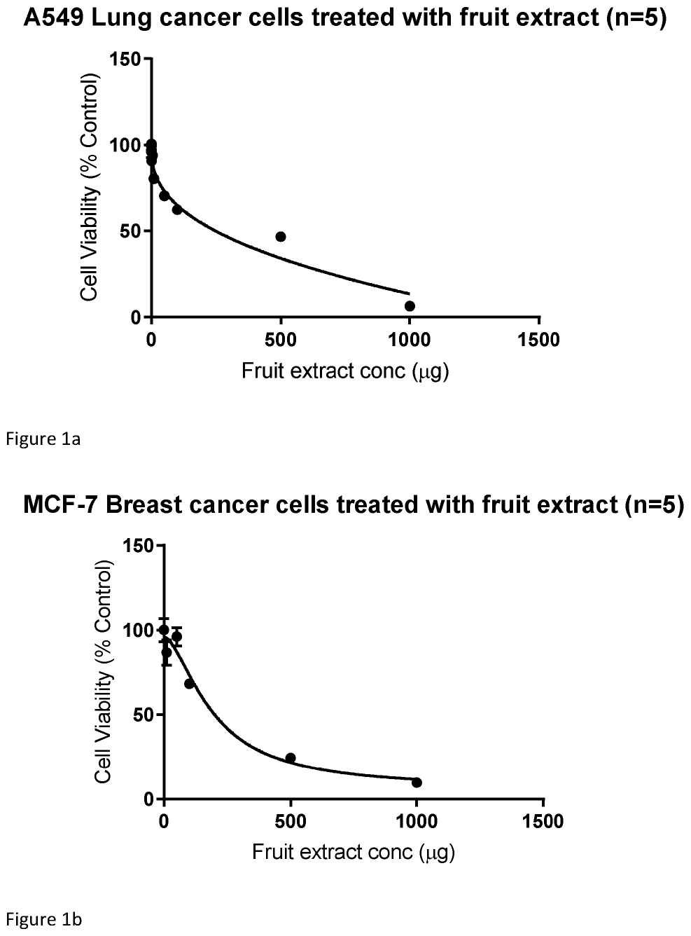

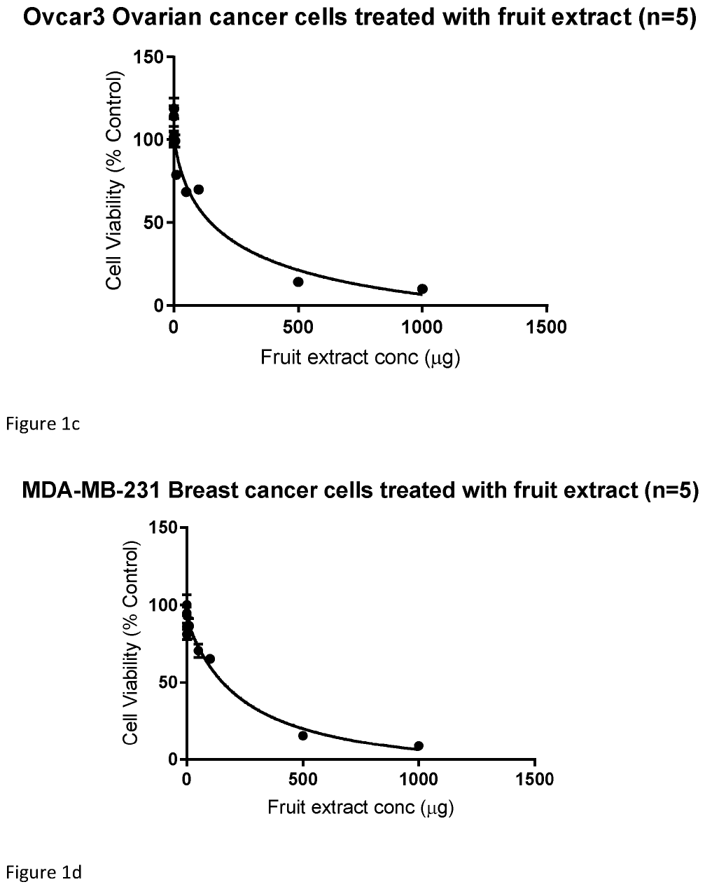

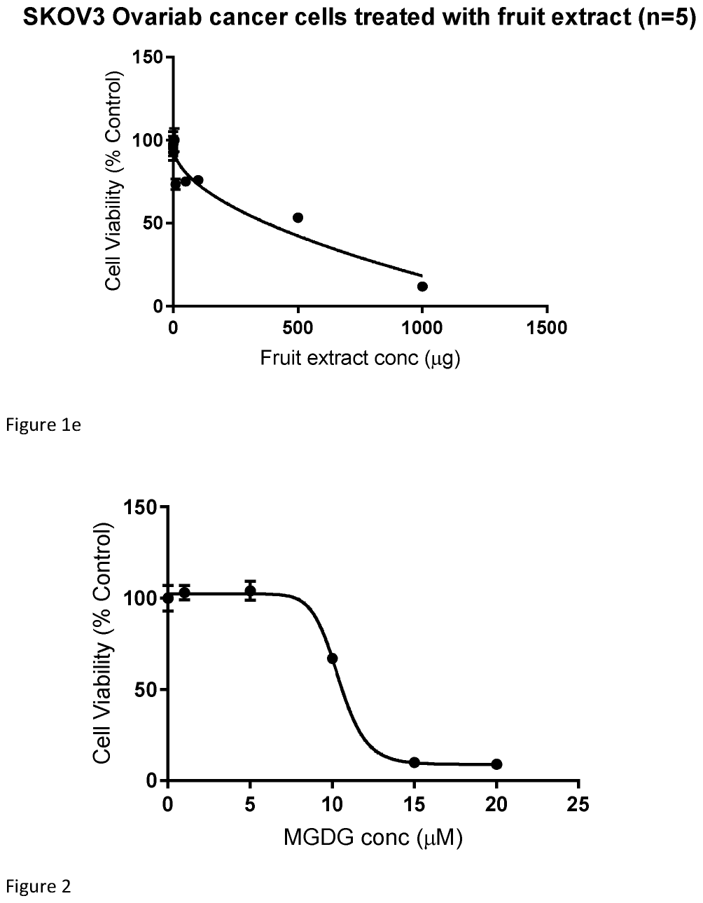

FIGS. 1a to e show the anti-proliferative activity of a crude fruit extract (manufactured using chloroform extraction method). Crude extract shows striking anti-cancer activity when tested against breast (FIGS. 1b and 1c), lung (FIG. 1a) and ovarian (FIGS. 1d and 1e) cancer cell lines.

FIG. 2 illustrates the anti-proliferative effects of MGDG extracted from tomatoes using HPLC: Cell line=Lung Carcinoma (A549), N=4, Error=S.E.M).

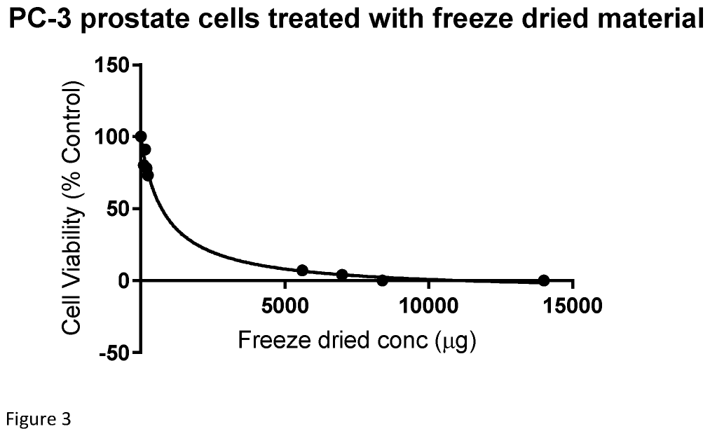

FIG. 3 demonstrates that on extract according to the invention yields bioavailable MGDG.

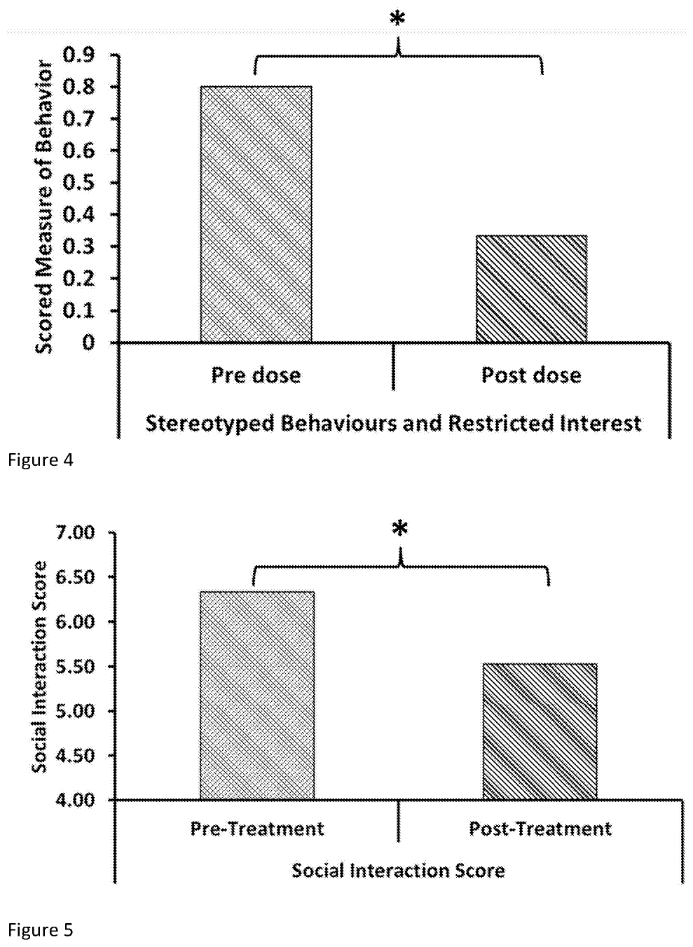

FIG. 4 show that treatment of cells with with fruit extracted from an ‘active’ line (with bioavailable MGDG) results in the suppression of translation, as observed in cells transiently transfected with a luciferase reporter (n=8, error=S.E.M).

FIG. 5 shows that treatment with fruit extract results in the selective inhibition of Neuroligin 1 (5′UTR) luciferase reporter. Cells were transfected with either Neuroligin 1 or Neuroligin 2 5′UTR firefly luciferase constructs and a Renilla control. The experimental data presented represents four biological repetitions. The reporter constructs were kindly provided by Professor Nahum Sonenberg (McGill University). Neuroligin 1 has been demonstrated to be a viable pharmacological target for the treatment of Autism spectrum disorder (Gkogkas et al, 2013. Nature, 17; 371-377).

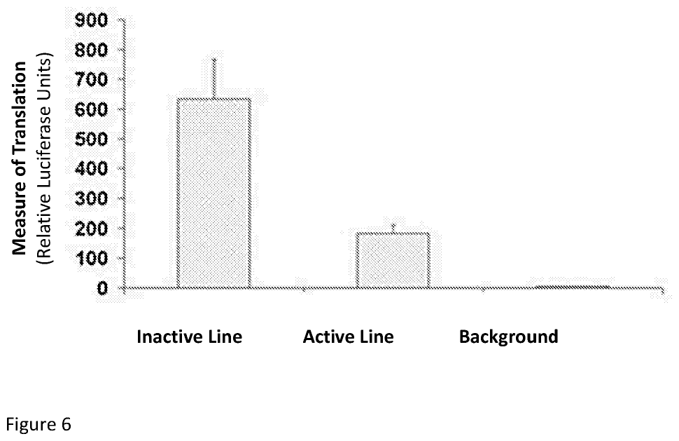

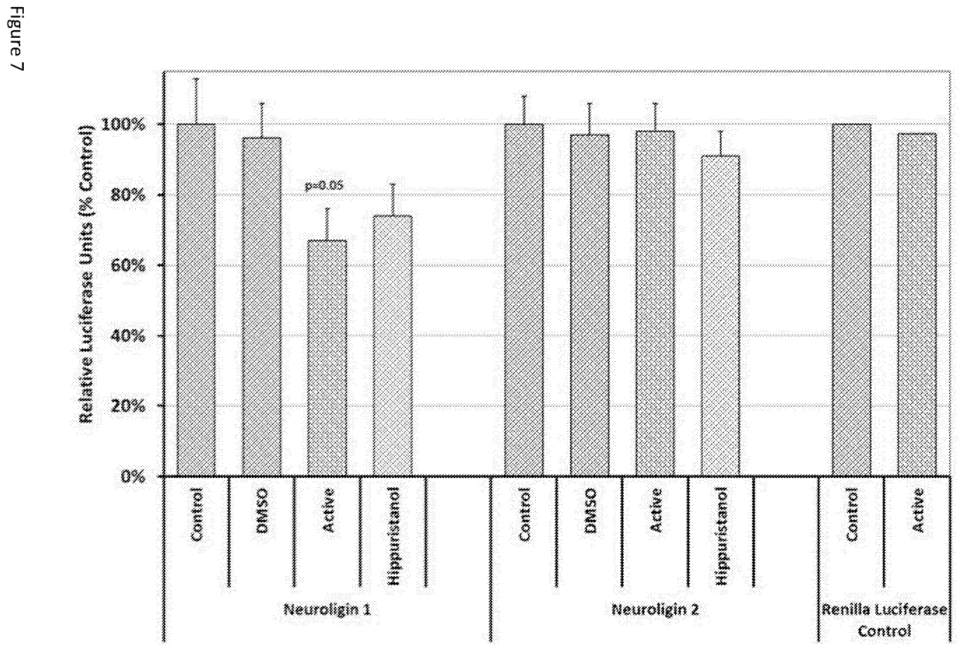

FIG. 6 illustrates statistically significant group wide (n=14) improvements in both stereotyped behaviour and social interaction after taking a low dose of nutraceutical according to the invention (2-way ANOVA, p=0.02).

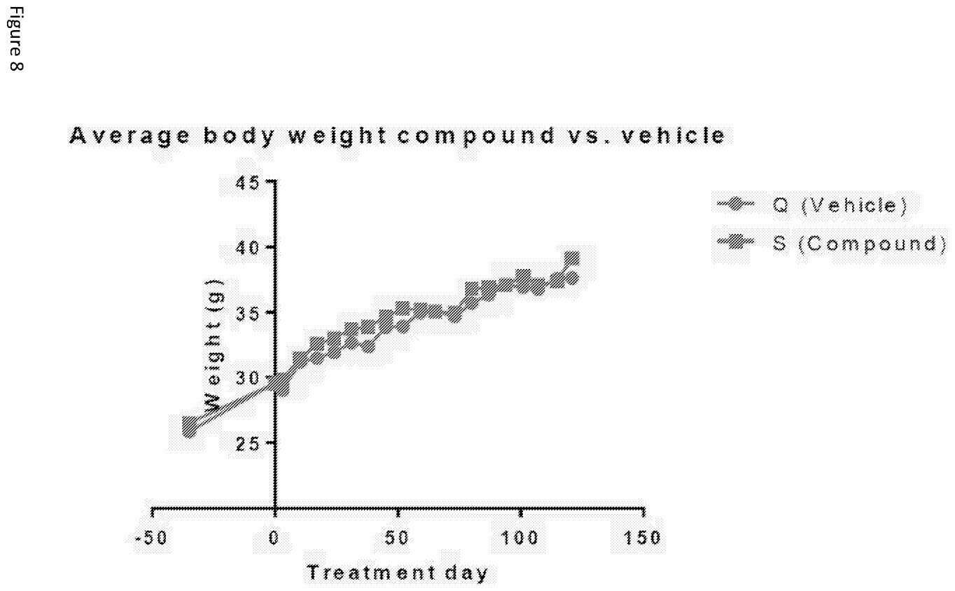

FIG. 7 illustrates statistically significant group wide (n=14) improvements in social interaction in ASD subjects administered with a nutraceutical according to the invention.

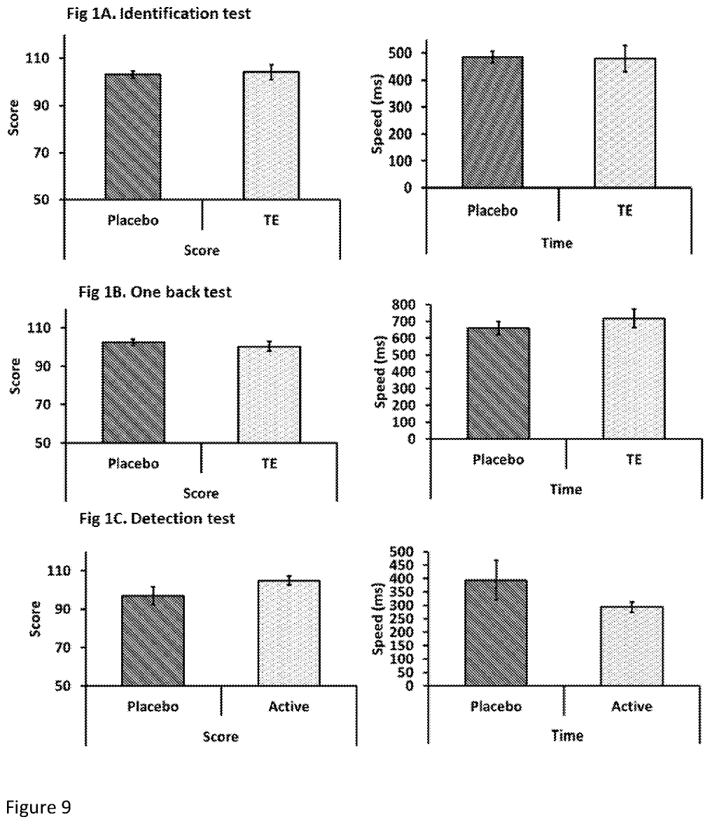

FIG. 8 illustrates that there are no adverse effects on mouse body weight from long term daily consumption of fruit extract according to the invention.

FIG. 9—shows that no effects were observed for either identification test scores (FIG. 9A) or for one back test times (FIG. 9C) after supplementation with MDGC containing tomato extract (TE). FIG. 9C shows that both detection scores and time showed some improvement in the TE group compared to placebo controls.

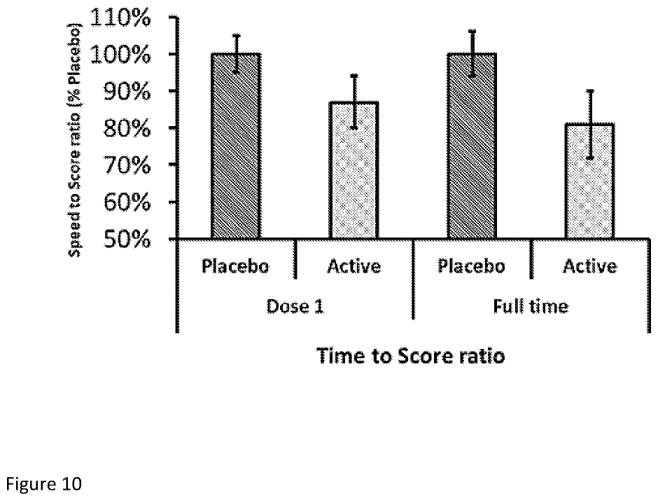

FIG. 10—shows that relative to the placebo control, improvements were noted in the tomato extract (TE) group for time to score ratios for the detection test. This was observed for both the tests conducted at rest (60 minutes dose) and for tests conducted after exercise (full time).

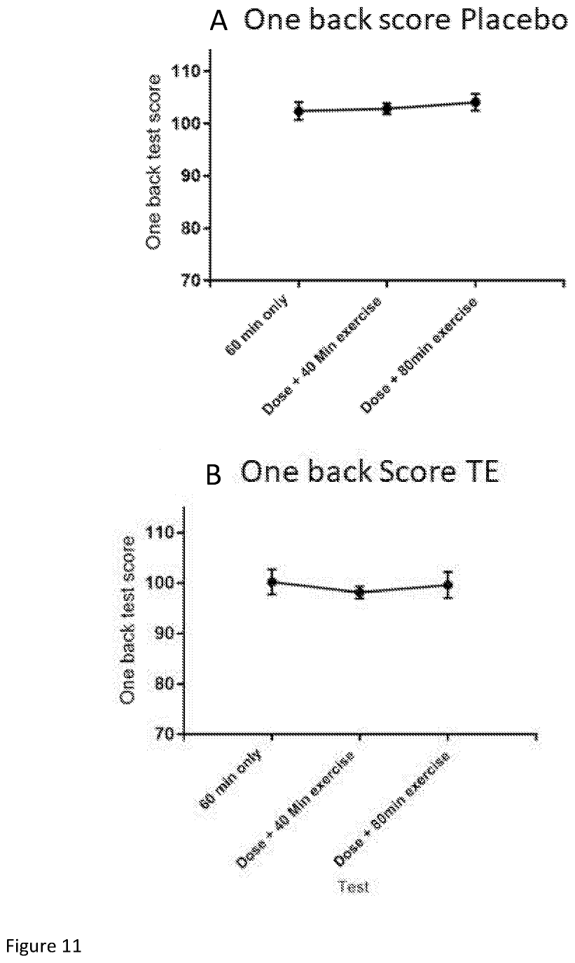

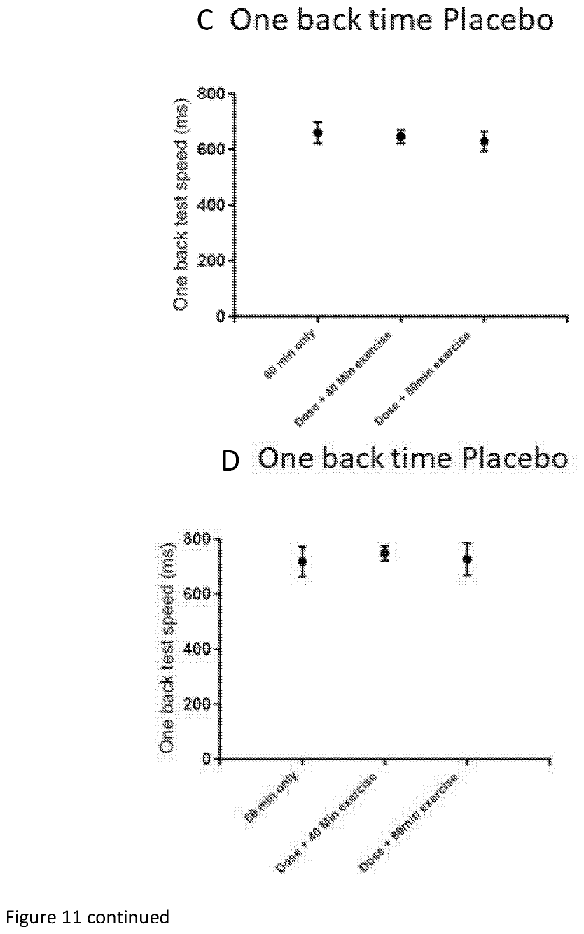

FIG. 11—demonstrates that the one back score is a reliable indicator of test setup ability. No difference could be detected in one back score (A) or time (B) in using any test design.

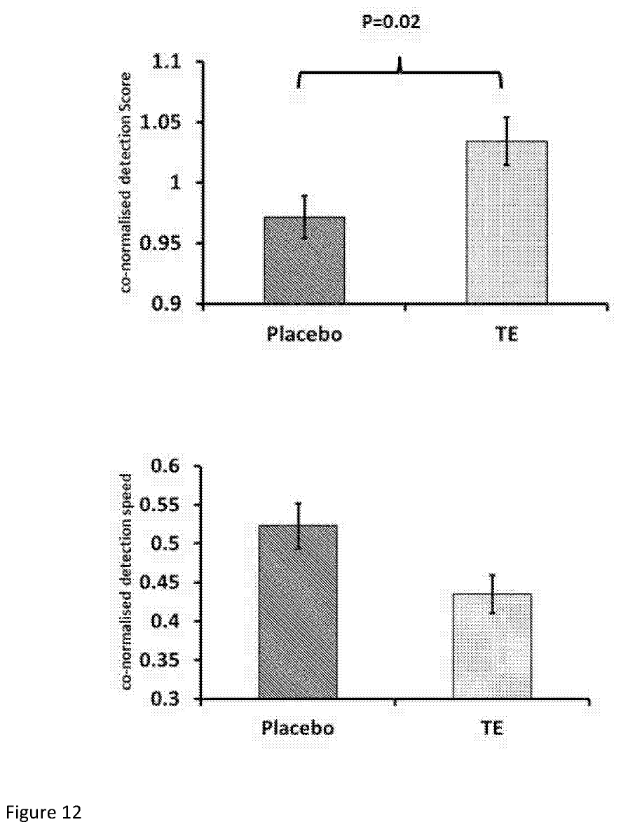

FIG. 12—shows that relative to the placebo control, the tomato extract (TE) group showed statistically significant improvements in both co-normalised (A) detection score and (B) detection time after exercise (P=0.02 and P=0.03 respectively).

Materials and Methods

Harvest—S. lycopersicum ‘M82’, Money maker and Green Envy tomatoes were grown in the UK under standard glasshouse conditions (16 h day length, day temp 22° C. and night temp 20° C.). Plants were grown in 7.5 litre pots of Pro C2 coarse potting compost (Levington). Irrigation supplemented with Vitax 214. One Kg of fresh fruit was harvested containing the correct ratio of metabolites as determined by volatile organic compounds and observed to contain 150 mgs MGDG.

Processing—harvested material was processed via high speed blending in a thermomix blender as per the manufacturer's instructions for 10 minutes at a continuous maximum speed. Samples were filtered to remove insoluble both debris and bacteria. Fruit pulp was then freeze dried and either re-suspended in DMSO (Sigma) at a concentration of 25 μg per μl or in RPMI 1640 media at a concentration of 28 μg per μl. Alternatively, the pulped material was snap frozen in liquid nitrogen, ground to a powder and then an extract manufactured using the solvent extraction method described below. By this method 1 kg of fruit could be processed into a powder in which there is 1 mg of MGDG per gram of powder.

Solvent extraction method—1 gram of snap frozen ground whole fruit was first suspended in 20 mls methanol and heated to 50° C. for 10 minutes, then filtered using mesh. Chloroform and water were then added to this mixture at a ratio of 2:2:2 (methanol:Chloroform:Water). The lower chloroform containing layer is then removed and dried down to a solid under vacuum. This extract can be suspended in a solvent for application. In the instance of tissue culture, this may be DMSO however ethanol is also a suitable vehicle.

HPLC purification—purification was performed by batch-wise reverse phase HPLC (Varian Prostar; Polaris 5 micron C18-A column (250 mm×10 mm); gradient elution 80% H2O 20% MeCN to 0% H2O 100% MeCN following the following method: 80% H2O 20% MeCN 2 min; 0% H2O 100% MeCN 20 min; 0% H2O 100% MeCN 48 min; 80% H2O 20% MeCN 50 min).

Nutraceutical fruit drink—40 grams (equivalent to about 40 mls) of fruit pulp containing 6 mgs MGDG, 120 μg glutamic acid, 1.2 mgs Malic acid and 3.16 g of alpha-tocopherol were mixed with 10 mls of water to produce a fruit drink.

Human tissue culture experiments—lung (A549), ovarian (OVCAR-3, SKOV), breast (MDA-MB-231) and prostate (PC-3) cancer cell lines were chosen to provide a broad profile of different cancer cell types and their response to treatment with the natural compound. Cell were seeded as a mono-layer in 96 well flat bottom Nunc tissue culture plates at a density of 10,000 cells per well and supplemented with RPMI 1640 media (Sigma Aldrich) containing 10% foetal calf serum, penicillin and streptomycin. Cells were then treated for 72 hours with a range of concentrations of MGDG either DMSO soluble, media soluble sample or the pure synthetic molecule. Four biological repetitions were tested for each dose. Activity of each treatment was then assessed using wst-1 reagent (Roche) as per the manufacturer's instructions. Data was visualised using a victor plate reader (Perkin Elmer) at a wavelength of 450 nm.

Transient transfection conditions and luciferase reporter constructs—Firefly luciferase reporter plasmids containing the 5′ untranslated regions (UTRs) of the genes Neuroligin 1 and Neuroligin 2 were a kind gift from Professor Nahum Sonenberg (McGill) and used as described by Gkogkas et al, 2013. Nature, 17; 371-377.

Cells were transfected using FuGene 6 (Roche) following the manufacturer's instructions. The activities of firefly and Renilla luciferase in lysates prepared from transfected cells were measured using a commercially available Luciferase reporter assay system (Promega) and light emission was measured over a 10 sec interval using either a TECAN luminometer. For each experiment described, data was obtained from a minimum of at least 3 biological repetitions per treatment.

Autism trail design—Participants—Fifteen adults (aged 8-53) diagnosed with Autism Spectrum Disorder (ASD) took part in the study. Participants were recruited from the Autism Research Team's past participant database and Autism support groups. The sample consisted of 14 men and 1 woman, with a mean age of 30.4 years.

Assessments—A number of standardised assessments were used to assess participant's performance before and after consuming the tomato drink, in both the control and experimental conditions. Please see brief descriptions of these assessments below.
Autism Diagnostic Observation Schedule (ADOS). The ADOS is an observational assessment of Autism Spectrum Disorder (ASD). It is a semi-structured, standardised assessment of communication, social interaction, play, and restricted and repetitive behaviours. It contains questions and activities that elicit behaviours directly related to a diagnosis of ASD.
Wechsler Abbreviated Scale of Intelligence (WASI). The WASI is a brief and reliable measure of individual's general intelligence, based on four subtests: vocabulary, similarities, block design, and matrix reasoning. It provides an assessment of an individual's verbal and non-verbal intelligence.
Touch Test. The touch test is a standardised measure, which uses hairs of differing thicknesses to detect the sensitivity threshold of individuals.

Example Products According to the Invention

Example #1 A nutraceutical fruit drink for the amelioration of the symptoms of Autism Spectrum Disorder. A drink was manufactured using 40 grams (equivalent to about 40 mls) of fruit pulp containing 6 mgs MGDG, 120 μg glutamic acid, 1.2 mgs Malic acid and 3.16 mg of alpha-tocopherol mixed with 10 mls of water. The final composition is a fruit drink of about 50 mls, and is intended that is administered once a day.

Example #2 A 500 to 750 mg tablet or capsule containing 6 mgs of MGDG provided either as freeze dried material or as a solvent based extract of a fruit pulp. The MGDG is formulated with beta cyclodextrin to provide some resistance in the GI tract. The tablet of capsule may be taken once a day to ameliorate the symptoms of autism spectrum disorders.

Example #3 A food additive for use as a cancer prophylactic in pets. Approximately 5 g of dehydrated tomato paste manufactured from 50 g of whole fruit containing about 6.6 mg of MGDG was added to a 300 g daily amount of pet food to act as a cancer preventative.

A 400 g tin of pet food to which has been added 16 g of pulped tomato according to the invention or 1.6 g of tomato paste according to the invention. The tin of pet food contains about 2.4 mg of MGDG.

In another example, freeze dried fruit material ground to a powder wherein 1 g of powder contains 1 mg of MGDG was added to food products.

Preferably the products are formulated such that the dose administered is 0.2 mgs kg for a human and 0.3 mgs per kg for a dog, thus a 70 kg person would require 14 mgs daily dose, and a 20 kg dog would require a 6.6 mgs daily dose would be needed.

Example # 4 A tablet formulation for the treatment of inflammation. A tablet was produced containing 500 mgs of freeze dried fruit powder according to the invention. The fruit extract was freeze dried with beta cyclodextrin to provide gastro resistance. The resulting tablets contain 500 μg of MGDG and are intended for administration as an anti-inflammatory agent for conditions aggravated by the Toll-Like Receptor 4 pathway.

Results

Preparation of a Tomato Extract Containing Bioavailable MGDG

Tomatoes were harvested and processed according to the method described herein and yielded up to about 150 mgs of bioavailable MGDG per kg of harvested fruit.

The bioavailable MGDG may be consumed in any appropriate form. For example, if it is to be consumed as a fruit drink where the whole fruit may be processed via high speed blending in a blender, the material may be used fresh or stored in aliquots for future use. The material may be stored at −20 degrees centigrade. Alternatively the material may be concentrated before storage, for example by pulping and evaporation to produce a paste. In another embodiment the extract may be freeze dried before use, for example the extract may be dried to produce powdered fruit containing about 1 mg MGDG per 1 g of powder.

Toxicity Studies on Naturally Occurring Bioavailable MGDG

Mouse in vivo studies demonstrated that naturally occurring MGDG recovered from tomatoes has very low toxicity. The data presented in FIG. 8 shows that mice orally administered water as a control or 2000 mg/kg of naturally recovered MGDG everyday for 14 days showed no difference in survival or weight

Use of Bioavailable MGDG Isolated from Tomatoes

Cancer Prophylactic

Due to the rapidly aging population, the rate of new instances of cancer in England alone is predicted to rise by 33% over the next ten years affecting an estimated. 299,000 people by 2020 (based on 2008 figures). In the United States, the medical cost of care associated with e.g. colorectal cancer and prostate cancer alone totals $26 bn (2010 figure—National Cancer Institute). Cancer is often fatal and treatment and care of patients can place a heavy burden on health services. Effective new cancer preventatives are therefore urgently required.

Cancer cells are rapidly dividing and utilise the protein synthesis machinery for both proliferation and metastasis. Importantly very early stage cancer cells require the synthesis of key proteins, which are used to establish vascularisation and tumour formation e.g. VEGF. These proteins may be regulated by selective inhibitors of protein synthesis. Early intervention or better still, daily administration of a non-toxic low dose of selective translational inhibitor may prevent ‘seed’ cancer cells from developing into rapidly dividing tumours.

Translational inhibitors are very potent known anticancer compounds. However, such inhibitors are rare, the most well-studied is hippuristanol which receives much attention because it is highly efficacious, yet it is made in vanishingly small quantities by a rare coral. We have identified that when accessible to the cell, MGDG possess these anticancer properties and is bio-available in a fruit with the correct chemical composition

The inventors have extensive in-vitro cell assay data which demonstrates the anti-proliferative activity of MGDG is effective against a range of cancer types. This is exemplified in FIG. 1 which shows efficacy against lung, breast and ovarian cancer cell lines. The data present shows the results with MGDG recovered by HLPC and the result with MGDG recovered from a crude pulp extract by using a solvent extraction method. The crude fruit extract was manufactured from 10 g weight fruit extract of cultivar M82 which yielded 95 mgs of crude extract containing 15.7 μg MGDG per mg extract. This extract has also been shown to be efficacious against human SKOV ovarian and MDA-MB-231 cells in a preclinical testing. The extract was manufactured using the chloroform method and samples then resuspended using DMSO as a vehicle.

Similar results were seen in prostate cancer cells when treated with non-extracted, non-purified freeze dried material, as illustrated in FIG. 3. This data demonstrates that freeze dried material according to the invention, which has not been processed or refined is bioactive, and that the cells can access the MGDG.

Autism

Autism Spectrum Disorder, Dose calculations based on cell culture experiments—Previous cell culture experiments designed to assay the effects of natural MGDG on the synthesis of Neuroligins show that treatment with a 10 μM dose is sufficient to elicit an about 35% reduction in the levels of Neuroligin 1, measured using a luciferase reporter system. This equates to a dose of 7.5 μg per ml in culture and 1.125 mgs per kg for a mouse. Using a dosage conversion calculator factor of 12 the required concentration of active for a 60 kg human would be 5.6 mgs compound (see Freireich et al, Cancer Chemotherapy Reporter, 1966, 50:219-244 for conversion factors). To achieve this dose consumption of approximately ˜1 gram of freeze dried material would be required.

Efficacy of MGDG Containing Tomato Extract in the Treatment of Autism Spectrum Disorder

Autism is a lifelong developmental disability that affects how people perceive the world and interact with others. Autism is a spectrum condition. All autistic people share certain difficulties, but being autistic will affect them in different ways. Some autistic people also have learning disabilities, mental health issues or other conditions, meaning people need different levels of support. All people on the autism spectrum learn and develop. Whilst there are currently no know cures for the condition, the use of naturally sourced bioavailable MGDG is demonstrated here to have a beneficial effect on individuals with autism.

The discussion and data herein presents both (1) a mechanism of action and (2) wider evidence that consumption of a fruit drink containing a bioavailable and dietary source of MGDG is beneficial to ameliorating the symptoms of autism and enhancing and maintaining healthy cognitive function. Specific short term benefits for applications such as sport likely include clarity of thought and cognitive performance under heightened levels of anxiety or stress. Benefits for healthy cognitive aging, mild cognitive impairment and the maintenance of the central nervous system are also hypothesised due to the mechanism of action.

(1) Core Mechanism of Action: The inventors have a clearly defined mechanism of action for MGDG, and present evidence here to show that fruit extract according to the invention, comprising bioavailable MGDG and characterised by certain metabolites, selectively modulates the activity of a component of the protein synthesis pathway (eukaryotic initiation factor-4AI (eIF4A). Enhanced levels of protein synthesis activity due to eIF4A have been linked to a number of negative conditions including the impairment of cognitive performance; while dampening eI4FA activity has been proposed to be of substantial benefits to both health and healthy aging.

(2) Regulation of translation at the synapse is key for healthy cognitive function. Dysregulation of translation of the key synaptic protein Neuroligin-1 results in difficulties in autism spectrum disorder including symptoms such as impaired social interaction and altered communication (see Gkogkas et al, Nature. 2013, 493:371-7). Proteins such as Neuroligins are controlled by translation and are important for the formation and regulation of connections known as synapses between neuronal cells in the brain; this is essential for the maintenance of the balance in the transmission of information from neuron to neuron. It is likely that Neuroligin-1 functions as an evolutionary fear response which impedes normal cognitive function through hyper stimulatory excitation of the synapse.

Importantly it is also now known that Neuroligin-1 protein level increase following pharmacological treatments that generate cellular oxidative stress (Staab et al, PLoS Genet. 2014, 10:e1004100); a stressor linked to anxiety and emotional stress (see Bouayed et al, Oxid Med Cell Longev. 2009, 2: 63-67).

(3) Cognitive Enhancer Differentiation—MGDG, and in particular naturally sourced MGDG, is distinct from cognitive enhancers such as nootropics or blended nutrient drinks; because the bioactive is clearly defined and is bio-available with a distinct mechanism of action. Products such as alpha-brain (onnit) are nutrient based and therefore limited in application and present with limiting side effects such as nausea and headaches.

Clinical testing of alpha-brain (n=63) funded by Onnit reported poor results after testing with alpha brain (See recent trial—Solomon et al, 2016—Hum Psychopharmacol Clin Exp, 31:135-143). Results show only very modest improvements in 1 out of 26 different tests designed to show cognitive improvement; some measures show deterioration compared with control e.g. BMVT trial 11 tests (placebo higher than treatment at both time points). The authors of the study conclude that they were (1) unable to draw conclusions on the mechanism of action and (2) the trial needed to be repeated before any conclusions on efficacy can be made. It is likely that Nootropics or formulations solely dependent on vitamin blends may have limited value where dietary intake is sufficient to meet bodily needs. The MGDG of the invention has a defined mechanism of action which matches defined applications

Experimental Evidence—MGDG extracted from tomatoes according to the invention are demonstrated herein to selectively control the synthesis of proteins in a tissue culture experiment. Specifically, neuroblastoma cells, a widely accepted cell culture model of neuronal cells, were transfected with a reporter assay designed to detect selective control of translation. Some of the cells were treated with MGDG containing tomato extract and a reduction in activity was only seen when MGDG was present (as indicated by the ‘active line’ bar in FIG. 4). No activity could be detected in other lines or shop bought fruit tested.

The reporter data presented in FIG. 5 suggests that certain galactolipids are able to restore the balance of Neurologin-1 protein synthesis, while not effecting the levels of Neuroligin-2 protein; Neuroligin-2 is required for continuous (healthy) maintenance of inhibitory synapses in the medial prefrontal cortex (Jiang et al, Molecular Psychiatry, 2015, 20:850-859). Dramatic overexpression of Neuroligin-1 has been demonstrated to induce cognitive dysfunction after injury as measured by decreasing neurological score (Shen et al, Stroke. 2015, 46:2607-2615), however it is likely that aberrant lower levels of this protein can also impair cognitive function in certain tests in healthy individuals.

FIG. 5 shows that the levels of Neuroligin 1, the protein that drives autism (see Gkogkas et al, 2013. Nature, 17; 371-377), can be controlled in a cell culture reporter luciferase assay model through treatment with naturally sourced MGDG. This experiment demonstrates that Neuroligin 1 is dependent on the activity of eIF4A (see column marked hippuristanol) and can be dampened through treatment with naturally sourced MGDG. Importantly levels of Neuroligin 2 reporter activity are unaffected by the MGDG; Neuroligin 2 is required for normal synaptic activity.

The effects of a single dose of a nutraceutical containing MGDG on fourteen ASD adults was investigated. Each individual received a fruit drink, as described earlier, containing 40 mls of pulped fruit (MGDG content ˜6 mgs). Tests were conducted by trained personnel 90 minutes after treatment. Preliminary data for the nutraceutical shows a statistically significant decrease in autistic symptoms (measured by the Autism Diagnostic Observation Schedule—ADOS); average 30% ADOS for one test group. In some instances, individuals benefit by up to 50% lower ADOS scores.

FIG. 6 demonstrates statistically significant group wide (n=14) improvements in both stereotyped behaviour and social interaction after taking low dose of MGDG n a nutraceutical drink (2-way ANOVA, p=0.02). Stereotyped behaviours and restricted interests examine sensory processing issues and interests like hand flapping and repetitive movements. This test also assesses whether the individual uses objects with purpose or not. An improvement in this symptom may result in a dramatic improvement in life quality.

FIG. 7 demonstrates statistically significant group wide (n=14) improvements in social interaction. This test measures elements like eye gaze and facial expression; if the participant responds to his or her name, and whether he or she uses a social smile at appropriate times.

Improvements in IQ and Visual Intelligence Testing (V-IQ)

The data presented below demonstrates that after taking a nutraceutical drink according to the invention, containing 6 mgs of naturally sourced MGDG, an improvement in IQ and visual intelligence is observed

Fifteen adults (aged 8-53) with autism took part in the study. The sample consisted of 14 men and 1 woman, with a mean age of 30.4 years. A single drink comprising 40 mls of pulped tomato juice was administered to test subjects. After consumption of the drink, the experimenter left 90 minutes before beginning the visual IQ test. Visual IQ test design is consistent with the Wechsler Abbreviated Scale of Intelligence test. No change in non-visual IQ was observed.

A team of psychologists undertook wide testing of two groups of individuals; a component of these tests includes a number of IQ tests. The results showed a group wide improvement in scores of 6% for verbal intelligence, with 75% response rates (9 out of 12 individuals showed at least some improvement in this measure). Some individuals benefited by up to 20% in this measure e.g. 73 improved to 88, and improvements were also recorded across the range of starting V-IQs e.g. improvement of 117 to 129 after treatment—the highest starting V-IQ in the test. Some individuals improved scores by up to 15% in combination IQ testing (verbal intelligence and non-verbal intelligence combined) e.g. from 79 to 91.

The ability of the naturally sourced MGDG to improve IQ and V-IQ is supported by published data relating to glycolipids which proves these are important molecules for the wider development and maintenance of the central nervous system (CNS). Galactolipids in the same class as MGDG form approximately 30% of the composition of myelin; myelination continues across many decades in the human and brain myelin has proven involvement in normal cognitive function, learning and IQ (for review see; Fields, Trends Neurosci. 2008, 31:361-370). Further, age-related myelin breakdown has been identified as a driver of cognitive decline (see Bartzokis, Neurobiol Aging. 2004, 25:5-18); through progressive disruption of neural impulse transmission that degrades the temporal synchrony of widely distributed neural networks underlying normal brain function.

Galactolipids are a known key ingredient in breast milk supporting cognitive development. Levels of the glycolipid chain fatty acid components of MGDG in breast milk during lactation are determined by the FADS1 and FADS2 gene (Xie, L. and S. Innis, 2008, J. Nut., 138:2222-2228), which in turn has been positively associated with higher levels IQ independent of social class, and maternal cognitive ability (see Caspi et al, Proc Natl Acad Sci USA. 2007, 20; 104); up to 7 IQ points. Further, treatment of rats with lead, known to impair neuronal function, induced decreased levels of galactolipids in brain tissue (Deng and Poretz, 2001).

Improvements in Sports Cognition

The data presented below demonstrates the ability of bioavailable MGDG in tomato extract (TE) to improve cognitive impairment after a period of strenuous exercise. In particular, the data shows that the TE can result in exercise linked cognitive enhancement as demonstrated by improved psychomotor ability.

In order to demonstrate the effect of TE containing MGDG on psychomotor ability subjects were tested for cognitive impairment after a period of strenuous activity having been administered TE containing MGDG or a placebo.

Materials and Methods

Participants—17 healthy recreational team sports players (age; 28.4±4.6 years, weight; 84.9±9.8 kg, height; 179.7±8.6 cm) provided written informed consent and participated in the study. All participants completed a medical screening questionnaire before testing began. In the days preceding the trial, participants were instructed to maintain a normal diet, and also asked to refrain from caffeine and alcohol consumption in the 24 hrs prior. Ethical approval was granted by the St. Mary's University ethics committee.

Supplementation—In a double-blind, randomised control trial, participants were randomly assigned to either a placebo or intervention group (tomato extract (TE)). Participants ingested a 3 g dosage of either the TE supplement containing about 18 mg of bioavailable MGDG (O'Kennedy et al., European Journal of Clinical Nutrition volume 71, pages 723-730 (2017)) or the placebo, with water 60 min prior to the commencement of the test.

Exercise Protocol—Participants completed an adapted version of the Bath University Rugby Shuttle Test (BURST). The BURST is a rugby union-specific match-play simulation protocol, designed to replicate the physical demands of elite rugby union forwards. The requirements of the exercise protocol have been detailed elsewhere (Roberts et al., 2010).

In brief, the adapted protocol comprised 8×300 s blocks followed by a 20 min “half time” period (rest) followed by a further 8×300 s blocks (total time 80 min). Each 300 s block consists of participants repeatedly performing shuttles of walking (20 m), cruising (20 m), jogging (10 m) and sprinting (10 m) which consists of a 1×maximum sprint (20 m) within the last 30 sec of each block.

Cognitive assessments—A 15 minute computerised cognitive test battery (CogState Ltd., Melbourne, Australia) was administered to all participants prior to, at half-time and following the adapted simulated rugby match protocol. CogState is a validated tool for measuring cognitive impairment induced by mental fatigue. The cognitive test battery included the following specific tasks:
Detection task measured reaction time, psychomotor function and information progression. Identification task measured reaction time and visual attention. One-back working memory measured visual learning and memory. Delayed recall task measured continuous retention and recall. Attention task measured the ability to maintain focused attention.

Performance was measured in terms of time or accuracy. Each task used playing cards as stimuli which are designed to have almost infinite equivalent alternative forms. A familiarisation or practice was included prior to each task. Once individuals are familiar with the test, it shows no practice effects.

Statistical Analysis—An analysis of covariance (ANCOVA) was used to compare the effects of both supplementation (tomato extract) and placebo on five key cognitive performance variables (accuracy, detection, identification, one card learning & one back time) over three time points (baseline, half-time and full-time), 95% confidence intervals are also described.

Results

17 healthy recreational team sports players were tested using a standard double-blind, randomised control trial. No adverse effects or side effects were reported after consumption of the TE material, either after dosing with no exertion or consumption of TE combined with exercise.

Testing 60 minutes after treatment—It was first sought to ascertain if treatment with an encapsulated tomato extract containing MGDG would provide enhanced cognitive function using a 15 minute computerised cognitive test battery provided by CogState (CogState Ltd., Melbourne, Australia). This established test package assesses performance in five key cognitive performance variables: accuracy, detection, identification, one-card-learning and one back time. It was found that there was no significant difference across a number of different test scores e.g. identification (TE 104.13±3.2 vs placebo 103.11±1.49), identification time (TE 480 ms±49.0 vs placebo 485 ms±21 ms) (see FIG. 9A), one back score one (TE 100.25±2.5 vs placebo 102.44±1.72), one back time (TE 718.63 ms±55.2 vs placebo 660.78 ms±39.17), card learning score (TE: 95.13±3.5 vs 99.89±2.25) or one card learning accuracy (TE 0.62±0.1 vs placebo 0.68±0.03).

However some improvement was observed in the detection test scores recorded for the treatment group (see FIG. 9B). A recorded average score difference for this test of 7.9 (combined standard error from both samples of 6.5). This represents an 8% improvement between groups. Response times were also lower in the TE group, comparing 394.2 ms±74 ms for the placebo group to 293 ms±18 ms for the TE group. The ratio of score to time for each individual also improved from 3.26±0.15 measured for the placebo group, compared to 2.83±0.22 for the TE treated group.

Testing after exercise—In order to determine any rescue of detrimental effects of exercise induced impairment of cognitive function, test subjects were subject to a range of predesigned controlled physical exertion (previously reported Bath University Rugby Shuttle Test (BURST)). Individuals were then tested for changes in cognitive responses for any effects of supplementation.

It was observed that collectively the TE group recorded a lower ratio of score to time for this test (see FIG. 10). The placebo group recording no change when compared to the non-exercise test (3.23±0.21), whereas the TE group score fell to (2.61±0.30). This represents a 19% improvement when comparing treatment and control after exercise.

Co-normalised detection score after exercise—The detection data was co-normalised for each individual to an appropriate internal test measure i.e. a test conducted using the same test platform that shows, no group wide aggregate difference; either between groups or changes in pre to post exercise (test chosen was one back test, see FIG. 11). Analysis of co-normalised data obtained after 80 minutes exercise (FIGS. 12A and 12B) identified statistically significant improvements in both detection tune and score (normalised score: TE 1.03±0.01 vs placebo 0.97±0.01 (p=0.02; Lower 95% CL 0.0075, Upper 95% CL 0.1184); normalised time: TE 0.43±0.02 vs placebo 0.52±0.03 (p=0.03; Lower 95% CL −0.1690, Upper 95% CL −0.0049). This represents a significant ˜6.5% higher score and a ˜17% better time after consumption of TE and after 80 minutes exercise.

Interestingly, for those individuals supplemented with TE, normalised detection times also measured better than the placebo group after 40 minutes exercise.

### SUMMARY

To summarise, the data presented herein demonstrates that administration of tomato extract (TE) containing MGDG had the following effects following exercise.
Psychomotor score: statistically significant 6.5% better normalised detection score for TE group compared to placebo (P=0.02) after 80 minutes exercise.
Psychomotor score: 4% better normalised detection score for TE group compared to placebo after 40 minutes exercise.
Psychomotor response time: statistically significant 17% better normalised detection time for TE group compared to placebo (P=0.03) after 80 minutes exercise.
Psychomotor response time: 8.5% better normalised detection time for TE group compared to placebo after 40 minutes exercise.
Psychomotor response time: 19% better time to Score Ratio for TE group compared to placebo after 80 minutes exercise.

It was also observed that the administration of TE containing MGDG had the following effect with no exercise but a 60 minute rest.
Psychomotor score: 8% better detection score for TE group compared to placebo after 60 minutes rest.
Psychomotor response time: 26% better detection time for TE group compared to placebo after 60 minutes rest.
Psychomotor response time: 13% better Speed to Score Ratio for TE group compared to placebo after 60 minutes rest.

In conclusion, we have identified that TE supplementation has a positive and statistically significant effect on co-normalised psychomotor detection scores after exercise. Also this effect appears to be linked to the duration of exercise at the point of testing. An effect is also observed without exercise but after a rest.

1-21. (canceled)

22. An extract of a fruit from the solanaceae family containing bioavailable MGDG, wherein MGDG has the formula of Formula I.

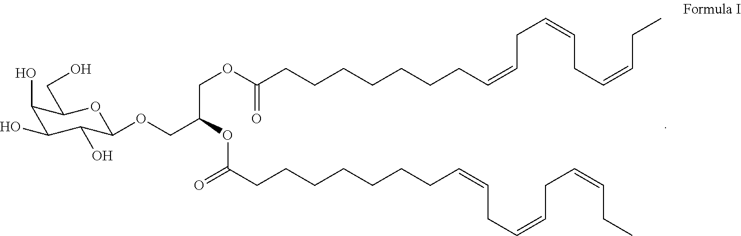 [SMILES: CC/C=C\C/C=C\C/C=C\CCCCCCCC(=O)OC[C@H](COC1OC(CO)C(O)C(O)C1O)OC(=O)CCCCCCC/C=C\C/C=C\C/C=C\CC]

23. The extract of claim 22, wherein containing the equivalent of at least 50 mg of bioavailable MGDG per kg of fruit.

24. The extract of claim 22, containing the equivalent of at least 100 mg of bioavailable MGDG per kg of fruit.

25. The extract of claim 22, provided as a pulp, a liquid, a paste or a dry powder.

26. The extract of claim 22, wherein the extract comprises pulped fruit containing at least 50 mg of bioavailable MGDG per kilogram of pulp.

27. The extract of claim 22, wherein the extract comprises a fruit paste containing at least 1 mg MGDG per 1 g of paste or containing between about 1 mg and about 5 mg MGDG per 1 g of paste.

28. The extract of claim 22, wherein the extract comprises a fruit powder containing at between about 2 mg and about 10 mg MGDG per 1 g of powder.

29. The extract of claim 22, wherein the extract contains metabolites in one or more of the following ratios:
a ratio glutamic acid to lutein of 0.00006 to 1 or less
a ratio of malic acid to lutein of 0.00069 to 1 or less
a ratio of alpha-tocopherol to lutein of 1.52 to 1 or less
a ratio of glutamic acid to citric acid of 0.02 to 1 or less
a ratio of malic acid to citric acid of 0.22 to 1 or less
a ratio of alpha-tocopherol to citric acid of 505 to 1 or less
a ratio of glutamic acid to pentose of 3.2 to 1 or less
a ratio glutamic acid to MGDG of 0.0005 to 1 or less
a ratio of malic acid to MGDG of 0.005 to 1 or less
a ratio of alpha-tocopherol to MGDG of 13 to 1 or less.

30. The extract of claim 22, wherein the fruit is a tomato.

31. A nutraceutical or pharmaceutical product comprising the extract of claim 22.

32. The nutraceutical or pharmaceutical product according to claim 31, wherein the composition is formulated for oral administration.

33. The nutraceutical or pharmaceutical product according to claim 31, containing the extract containing the equivalent of at least 50 mg of bioavailable MGDG per kg of fruit.

34. The nutraceutical or pharmaceutical product according to claim 31, containing between about 2 mg and about 20 mg of MGDG.

35. A method of treating and/or preventing one or more of autism spectrum disorder (ASD), cancer and inflammation, and/or for improving sports cognition, in particular for improving psychomotor function, comprising administering an effective amount of an extract according to any of claim 22.

36. A method of obtaining bioavailable MGDG from fruit comprising harvesting the fruit when the MGDG is in the bioavailable form, wherein the method comprises the steps of:
determining the level in the fruit of two or more of the following metabolites glutamic acid, lutein, malic acid, alpha-tocopherol, pentose and citric acid;
comparing the relative levels of the two or more metabolites;
comparing the observed relative levels to known relative levels and harvesting the fruit when relative levels indicative of high levels of bioavailable MGDG are observed.

37. The method of claim 36, wherein if one or more of the following ratios are observed in a fruit, the fruit is ready to pick and the levels of bioavailable MGDG will be at least about 60 to about 300 mg per kg of fruit:
a ratio of glutamic acid to lutein of 0.00006 to 1 or less
a ratio of malic acid to lutein of 0.00069 to 1 or less
a ratio of alpha-tocopherol to lutein of 1.52 to 1 or less
a ratio of glutamic acid to citric acid of 0.02 to 1 or less
a ratio of malic acid to citric acid of 0.22 to 1 or less
a ratio of alpha-tocopherol to citric acid of 505 to 1 or less
a ratio of glutamic acid to pentose of 3.2 to 1 or less
a ratio glutamic acid to MGDG of 0.0005 to 1 or less
a ratio of malic acid to MGDG of 0.005 to 1 or less
a ratio of alpha-tocopherol to MGDG of 13 to 1 or less.

38. The method of claim 36, wherein if one or more of the following absolute levels are observed in a fruit, the fruit is ready to pick and the levels of bioavailable MGDG will be at least about 60 to about 300 mg per kg of fruit:
level of glutamic acid per gram of about 0.003 μg/mg or less
level of malic acid per gram of about 0.03 μg/mg or less
level of alpha-tocopherol per gram of about 79 μg/mg or less

39. The method of claim 36, wherein the fruit is a tomato.

40. A method of harvesting fruit of the solanaceae family, wherein a kilogram of harvested fruit contains at least 60 mg of bioavailable MGDG, wherein the method comprises:
obtaining fruit;
determining the odour fingerprint by assaying for one or more of the following volatile organic compounds: beta-ionone, hexanal, beta-damascenone, 1-penten-3-one, 2-methylbutanal, trans-2-hexenal, isobutylthiazole, 1-Nitro-2-phenylethane, trans-2-heptenal, phenylacetaldehyde, 6-methyl-5-hepten-2-one, cis-3-hexanol, 2-Phenylethanol, 3-methylbutanol and methyl salicylate; and
selecting those fruit with the optimal odour fingerprinting.

41. The method of claim 40, wherein the fruit is a tomato.
